(19) United States
(12) Patent Application Publication
(10) Pub. No.: US 2019/0255229 A1
(43) Pub. Date: Aug. 22, 2019

(54) ADIPOSE TISSUE SIZING SYSTEMS AND METHODS

(71) Applicant: Roger S. Hogue, Maple Grove, MN (US)

(72) Inventor: Roger S. Hogue, Maple Grove, MN (US)

(21) Appl. No.: 16/279,852

(22) Filed: Feb. 19, 2019

Related U.S. Application Data
(60) Provisional application No. 62/632,147, filed on Feb. 19, 2018.

(51) Int. Cl. A61M 1/00; G01N 15/02

(52) U.S. Cl. CPC A61M 1/008; A61M 1/0086; A61M 2205/7545; G01N 15/0272; A61M 2202/08; A61M 1/0001

(57) ABSTRACT

An adipose tissue sizing device includes a first tube having a hollow inner portion, a sidewall radially extending around the hollow inner portion, a first end, and a second end opposite the first end. An inlet interface in fluid communication with the hollow inner portion of the first tube is configured to receive adipose tissue particles, and to allow the adipose tissue particles to pass into the hollow inner portion of the first tube. A filtering assembly, having a plurality of apertures therein, is positioned in the hollow inner portion of the first tube in a flow pathway of the adipose tissue particles, and is configured to resize the adipose tissue particles passing through the apertures according to a size of the apertures. An outlet interface in fluid communication with the hollow inner portion of the first tube is configured to allow the resized adipose tissue particles to pass out of the hollow inner portion of the first tube.

### CROSS-REFERENCE TO RELATED APPLICATION(S)

This application claims priority to U.S. Provisional Application No. 62/632,147 filed on Feb. 19, 2018 for “Adipose Tissue Sizing Systems and Methods” by R. Hogue, which is hereby incorporated by reference in its entirety.

### BACKGROUND

### Field

Various embodiments disclosed herein relate to adipose tissue sizing.

### Description of Related Art

Adipose tissue, or body fat, is loose connective tissue composed mostly of adipocytes, such as fat cells, along with a vast array of regenerative cell populations, including adipose-derived stem cells or mesenchymal stem cells, which have tremendous potential benefits for human tissue regeneration.

In order to harvest adipose tissue or adipose tissue containing regenerative call populations such as adipocyte-derived stem cells, a minimally-invasive treatment that uses tumescent liposuction techniques to harvest fat tissue can be used. Additional processing steps are routinely used following the initial lipoharvesting procedure (i.e., tumescent liposuction), including fat aspirate particle sizing (micronizing), fat aspirate particle concentration via centrifugation in order to create an autologous fat graft that can be used for injection or deployment during an autologous fat grafting (fat transfer) treatment for the purpose of aesthetic (cosmetic) and/or regenerative purposes. Autologous fat grafting and/or autologous regenerative treatments containing autologous fat aspirate particles are used for cosmetic and/or therapeutic rejuvenation, restoration, and repair of aging or degenerative tissues such as the skin, hair, face, body, breasts, cleavage, dorsum of hands, soft tissue, wounds, scars, musculoskeletal tissues, and genitalia.

Currently, several procedures exist for sizing fat aspirate particles. One such procedure involves placing the fat inside a chamber having many small steel balls immersed in saline. The chamber is then shaken whereby the steel balls macerate the fat while the saline cleans it. This procedure can result in indiscriminate sizing of fat particles due to the high variability in shaking the chamber. Other procedures entail passing the fat aspirate across a mesh-like surface or screen with a square-shaped pattern to size the particles using luer-to-luer syringe transfer. This processing can damage the fat aspirate particle and be time consuming and physically straining. As a result, there is a need for systems and methods that result in precise outer dimensional sizing contemporaneously with lipoharvesting and/or post-liposuction processing of fat aspirate particles for cosmetic and/or regenerative purposes.

### SUMMARY

An adipose tissue sizing device includes a first tube elongate along a first direction, having a hollow inner portion, a sidewall radially extending around the hollow inner portion, a first end, and a second end opposite the first end. An inlet interface is in fluid communication with the hollow inner portion of the first tube, and is configured to receive adipose tissue particles, and to allow the adipose tissue particles to pass into the hollow inner portion of the first tube. A filtering assembly, having a plurality of apertures therein, is positioned in the hollow inner portion of the first tube in a flow pathway of the adipose tissue particles, and is configured to resize the adipose tissue particles passing through the apertures according to a size of the apertures. An outlet interface is in fluid communication with the hollow inner portion of the first tube, and is configured to allow the resized adipose tissue particles to pass out of the hollow inner portion of the first tube.

In some embodiments, the filtering assembly includes a second tube having the plurality of apertures in a radially outer surface thereof, the second tube having a first end coupled to the inlet interface and a second end coupled to the outlet interface, and being positioned in the hollow inner portion of the first tube.

A dual-stage liposuction system includes a tissue harvesting tool configured for harvesting adipose tissue particles from a patient, an adipose tissue sizing device having its inlet interface coupled to the tissue harvesting tool for receiving the harvested adipose tissue particles, and a container coupled to an outlet interface of the adipose tissue sizing device to receive resized adipose tissue particles therefrom, the container being couplable to a pump to provide suction to aspirate the adipose tissue particles through the system.

Any of the features of each embodiment can be applicable to all aspects and embodiments identified herein. Moreover, any of the features of an embodiment is independently combinable, partly or wholly with other embodiments described herein in any way (e.g., one, two, three, or more embodiments may be combinable in whole or in part). Further, any of the features of an embodiment may be made optional to other aspects or embodiments. Any aspect or embodiment of a method can be performed by a system or apparatus of another aspect or embodiment, and any aspect or embodiment of a system can be configured to perform a method of another aspect or embodiment.

### BRIEF DESCRIPTION OF THE DRAWINGS

These and other features, aspects, and advantages are described below with reference to the drawings, which are intended to illustrate, but not to limit, the invention. In the drawings, like reference characters denote corresponding features consistently throughout similar embodiments.

FIG. 1 illustrates a perspective view of an adipose tissue sizing device, according to some embodiments.

FIG. 2 illustrates a perspective view of an adipose tissue sizing device with the side luer port tubes removed to thereby expose the side luer ports, according to some embodiments.

FIG. 3 illustrates an exploded perspective view of an adipose tissue sizing device, according to some embodiments.

FIG. 4 illustrates a perspective view of an adipose tissue sizing device with the tube removed thereby exposing internal components, according to some embodiments.

FIGS. 5a, 5b, and 5c illustrate various views of a tube of the adipose tissue sizing device, according to some embodiments.

FIGS. 6a and 6b illustrate various views of a cap of the adipose tissue sizing device, according to some embodiments.

FIGS. 7a, 7b, 7c, and 7d illustrate various views of a chamber tube of the adipose tissue sizing device, according to some embodiments.

FIGS. 8a and 8b illustrate various views of a flat filtering disk of the adipose tissue sizing device, according to some embodiments.

FIGS. 9a and 9b illustrate various views of a luer port tube of the adipose tissue sizing device, according to some embodiments.

FIG. 10 illustrates a perspective view of an embodiment of an adipose tissue sizing device, according to some embodiments.

FIG. 11 illustrates a perspective view of another embodiment of an adipose tissue sizing device, according to some embodiments.

FIG. 12 illustrates a perspective view of another embodiment of an adipose tissue sizing device, according to some embodiments.

FIG. 13 illustrates a perspective view of another embodiment of an adipose tissue sizing device, according to some embodiments.

FIG. 14 illustrates a perspective view of another embodiment of an adipose tissue sizing device, according to some embodiments.

FIG. 15 is a diagram illustrating a further embodiment of an adipose tissue sizing device, according to some embodiments.

FIG. 16 is a diagram illustrating a further alternative embodiment of an adipose tissue sizing device, according to some embodiments.

FIG. 17 is a diagram illustrating a two-stage, combined lipoharvesting (using liposuction cannula) and adipose tissue sizing system, according to some embodiments.

### DETAILED DESCRIPTION

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### Filter Disk Embodiment(s)

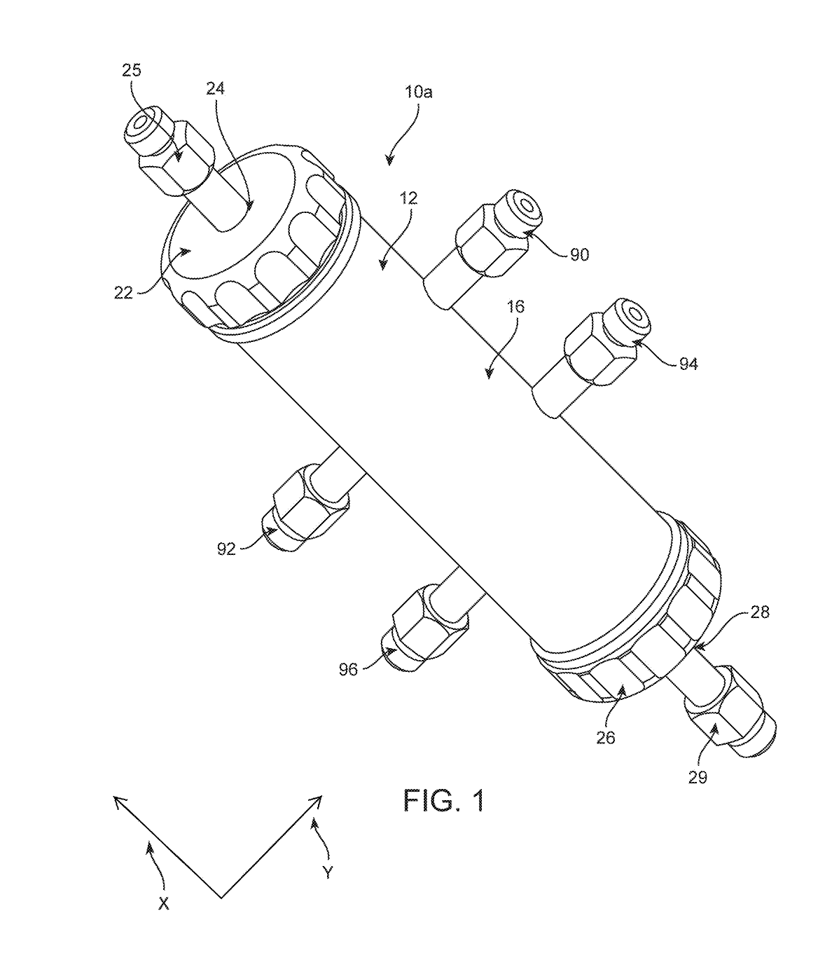

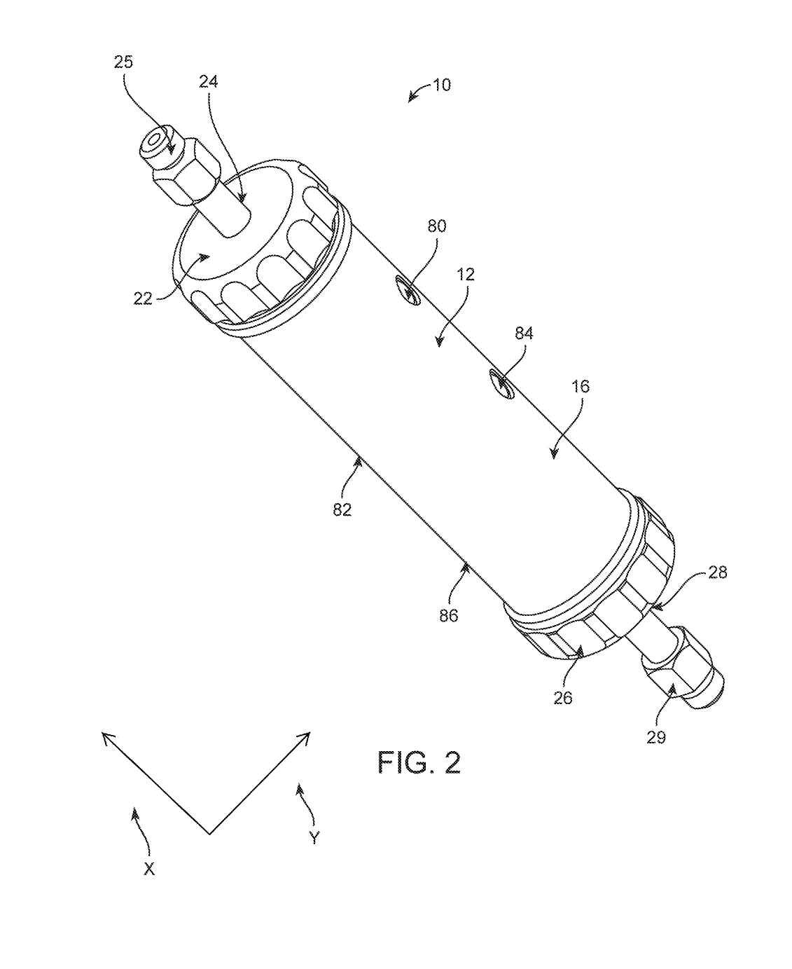

As shown in FIGS. 1 and 2, the disclosure includes an aspirate tissue sizing device 10 that can macerate and size fat aspirate particles into a variety of desired sizes. The device 10 includes a tube 12 that receives the fat aspirate particles and thereby sizes the particles accordingly. In order to size the particles, the device 10 includes a variety of components structurally oriented in order to achieve desired particle size.

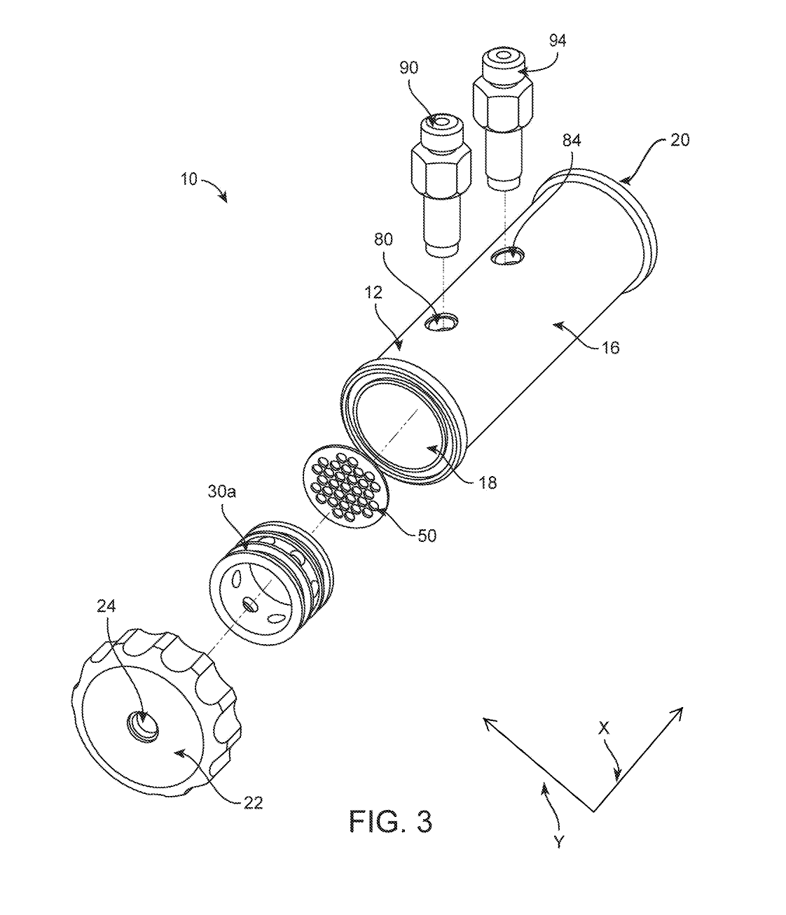

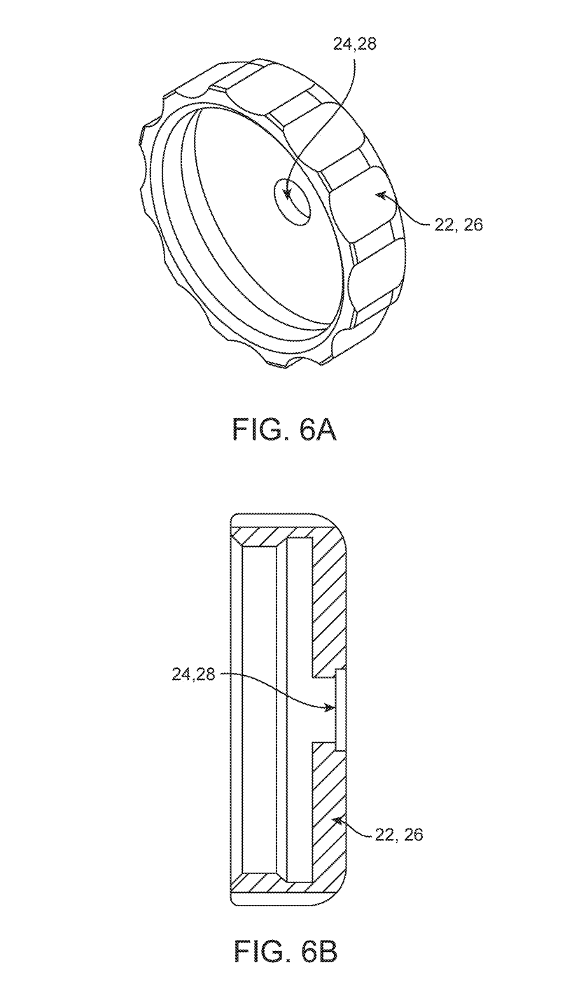

According to FIG. 3, the tube 12 includes a hollow inner portion 14, a sidewall 16 radially extending around the hollow inner portion 14, a first open end 18, and a second open end 20 that is opposite the first open end 18. As shown in FIGS. 6a and 6b, the device 10 includes a first cap 22 coupled to the first open end 18 whereby the first cap 22 may include an inlet luer port 24 in fluid communication with the hollow inner portion 14 of the tube 12. The first cap 22, via the inlet luer port 24, may act as an entryway for fat aspirate particles to enter the tube 12 for maceration and sizing. The device 10 also includes a second cap 26 coupled to the second open end 20 of the tube 12. Accordingly, the second cap 26 may include a outlet luer port 28 that is also in fluid communication with the hollow inner portion 14. The second cap 26, via the outlet luer port 28, may act as an exit for appropriately sized fat aspirate particles to exit the device 10.

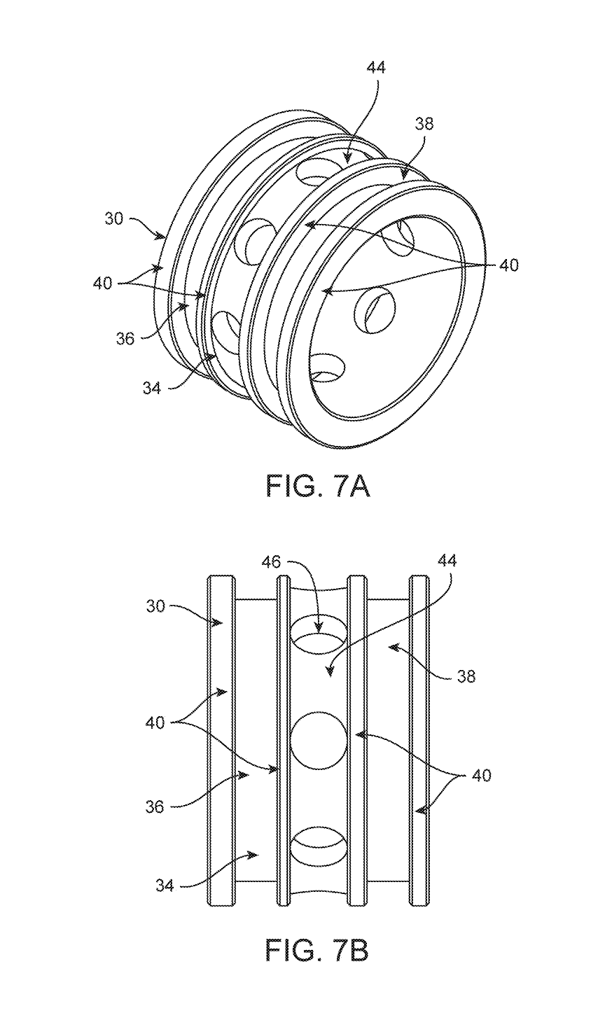

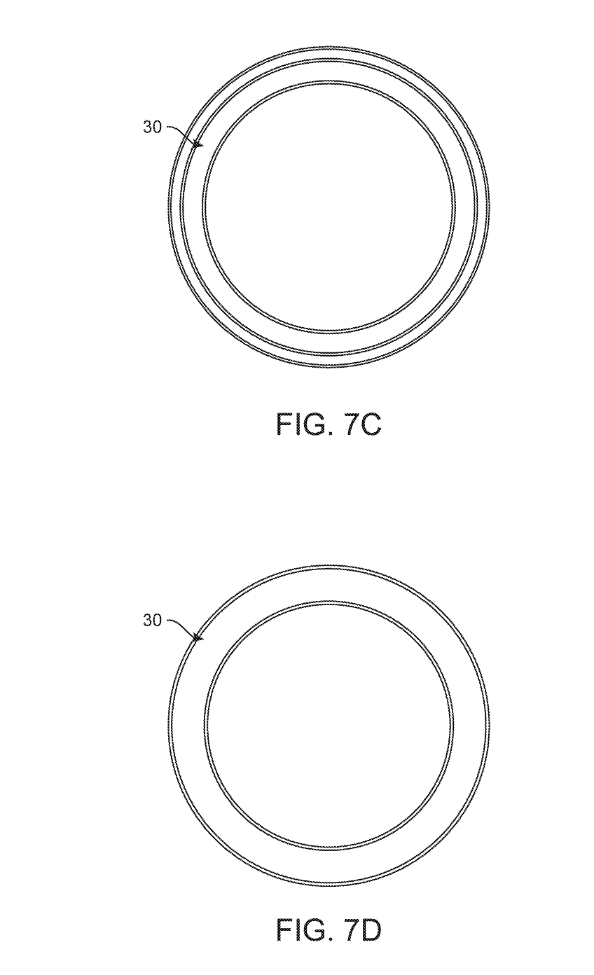

In order to size the fat aspirate particles, the device 10 may include various components located within the tube 12. Now, with reference to FIGS. 3, 4, 7a, 7b, 7c, and 7d, the device 10 may include two or more chamber tubes 30 each slideably received within the hollow inner portion 14 of the tube 12. Each chamber tube 30 is hollow and includes a chamber sidewall 34 that radially extends around the hollow part of the chamber tube 30 thereby allowing fat aspirate particles to travel through the chamber tube 30 and to continue to travel through the tube 12. Additionally, the chamber tube 30 includes a first portion 36 and a second portion 38 each having end walls 40 radially extending from the chamber sidewall 34. The end walls 40 may be arranged and configured to receive one or more seals 42 in each of the first and second portions 36, 38. The seal 42 may be arranged and configured to seal against an outer surface of the chamber tube 30 and an inner sidewall of the tube 12 to prevent liquid and fat from passing across the seal 42. This may ensure that all fat and liquid is forced to pass through the flat filtering disks 50 for appropriate sizing.

Furthermore, the chamber 30 may also include a middle portion 44 located between the first and second portions 36, 38. Within the middle portion 44, the chamber tube 30 may have a plurality of tube apertures 46 that are radially spaced apart from each other. Any one of the apertures of the plurality of tube apertures 46 may serve as an entry or exit point for fat aspirate particles to enter or exit the side of the device 10.

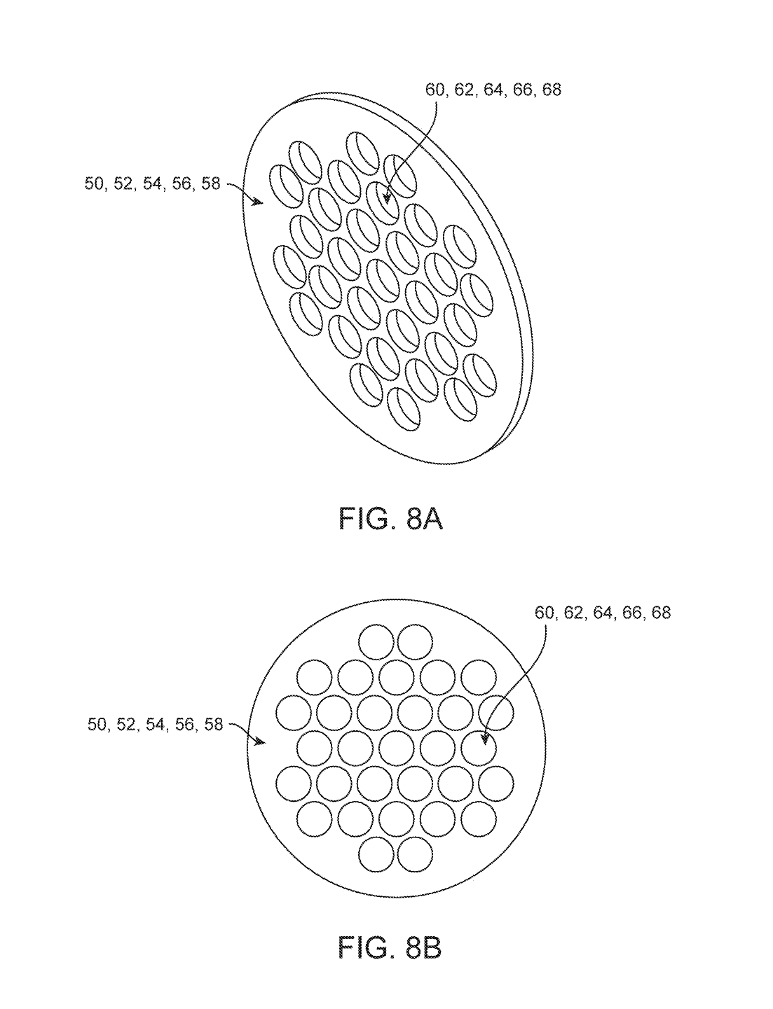

Now with reference to FIGS. 8a and 8b, to appropriately size the fat aspirate particles, the device 10 includes a filtering assembly, which may include at least one flat filtering disk 50, 52, 54, 56, 58 slideably received within the hollow inner portion 14 of the tube 12 whereby it is located, or sandwiched, between two chamber tubes 30. The flat filtering disk 50, 52, 54, 56, 58 includes a plurality of disk apertures 60, 62, 64, 66, 68 each substantially defining an aperture diameter 70, 72, 74, 76, 78. When fat aspirate particles are pushed into the device 10, via the inlet luer port 24, the fat aspirate particles travel through the tube 12 whereby the particles are forced through the plurality of disk apertures 60, 62, 64, 66, 68. In doing so, the fat aspirate particles are reduced in size to approximately the diameter of the aperture diameter 70, 72, 74, 76, 78. In some embodiments, the aperture diameter 70, 72, 74, 76, 78 is approximately equal to 2.0 millimeters, 1.6 millimeters, 1.3 millimeters, 1.1 millimeters, 0.9 millimeters, 0.8 millimeters, 0.6 millimeters, 0.4 millimeters, 0.3 millimeters, 0.2 millimeters, any dimension less than 0.2 millimeters, any dimension greater than 2.0 millimeters, any dimension between any of the abovementioned dimensions, and the like. Generally, it should be appreciated that the aperture diameter 70 may be any size diameter.

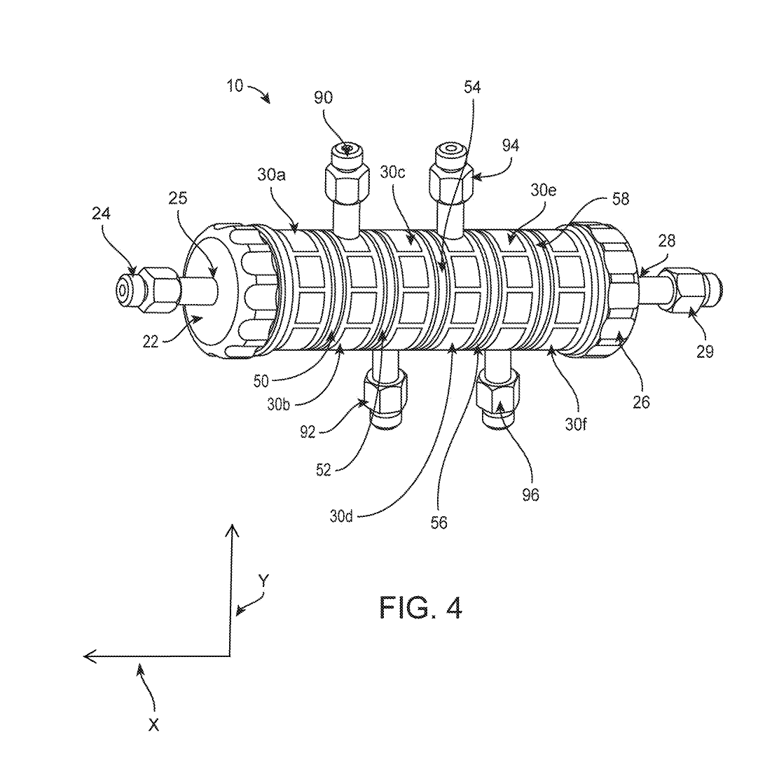

While the device 10 may have primary entry and exit points at luer ports 24, 28, the device 10 may also include luer ports located along the side of the tube 12. As illustrated in FIGS. 2, 3, 5a, 5b, and 5c, the device 10 may include zero or more side luer ports, which can serve as egress points for fat aspirate particles to exit the device 10. The benefit of side luer ports becomes apparent when looking at FIG. 4. According to FIG. 4, the device 10 may be equipped with a plurality of flat filtering disks 50, 52, 54, 56, 58, each having different aperture diameters in descending size from the first flat filtering disk 50 to the second flat filtering disk 52 to the third flat filtering disk 54 to the fourth flat filtering disk 56 to the fifth flat filtering disk 58. For example, the first flat filtering disk 50 may have aperture diameters of 2.0 millimeters, the second flat filtering disk 52 may have aperture diameters of 1.6 millimeters, the third flat filtering disk 54 may have aperture diameters of 1.3 millimeters, the fourth flat filtering disk 56 may have aperture diameters of 1.1 millimeters, and the fifth flat filtering disk 58 may have aperture diameters of 0.9 millimeters. So if the desired fat aspirate particle size is 1.1 millimeters, then the fat aspirate particles may egress the device 10 at the fourth luer port 86, via the fourth luer port tube 96, which is located directly downstream of the fourth flat filtering disk 56 having the desired size aperture diameters. If the desired fat aspirate particle size is 0.9 millimeters, then the fat aspirate particles may egress the device 10 at the outlet luer port 28. This will allow users the flexibility to size fat aspirate particles without having to disassemble the device 10 to install different size apertures or even size the fat aspirate particles through the use of multiple devices.

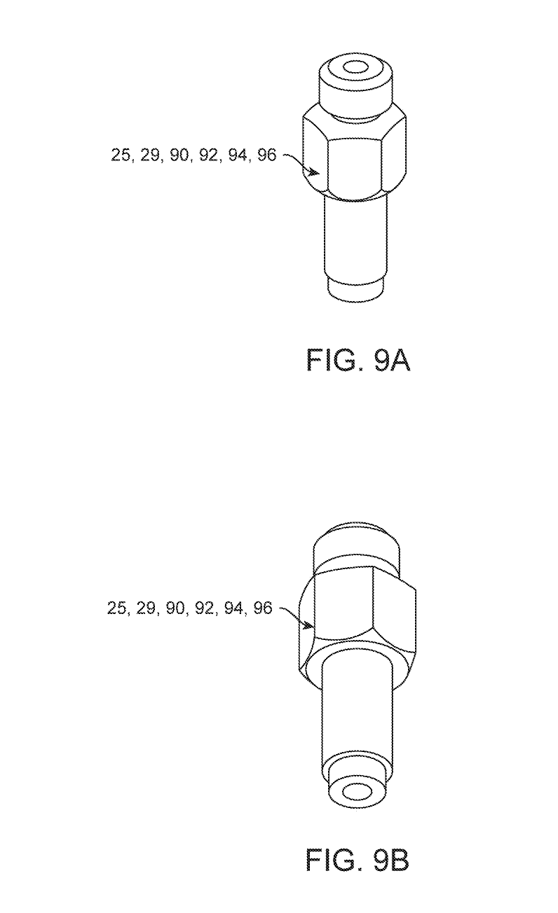

As illustrated in FIGS. 9a and 9b, the device 10 may also include one or more luer port tubes, which can be mechanically coupled to any of the luer ports, such as inlet luer port 24, outlet luer port 28, first luer port 80, second luer port 82, third luer port 84, and fourth luer port 86, to thereby allow the device 10 to be coupled to a secondary device, such as a syringe or any device having a luer fitting. Various size luer port tubes, length and diameter, may be used so that the device 10 may be sized and configured to fit to a variety of secondary devices.

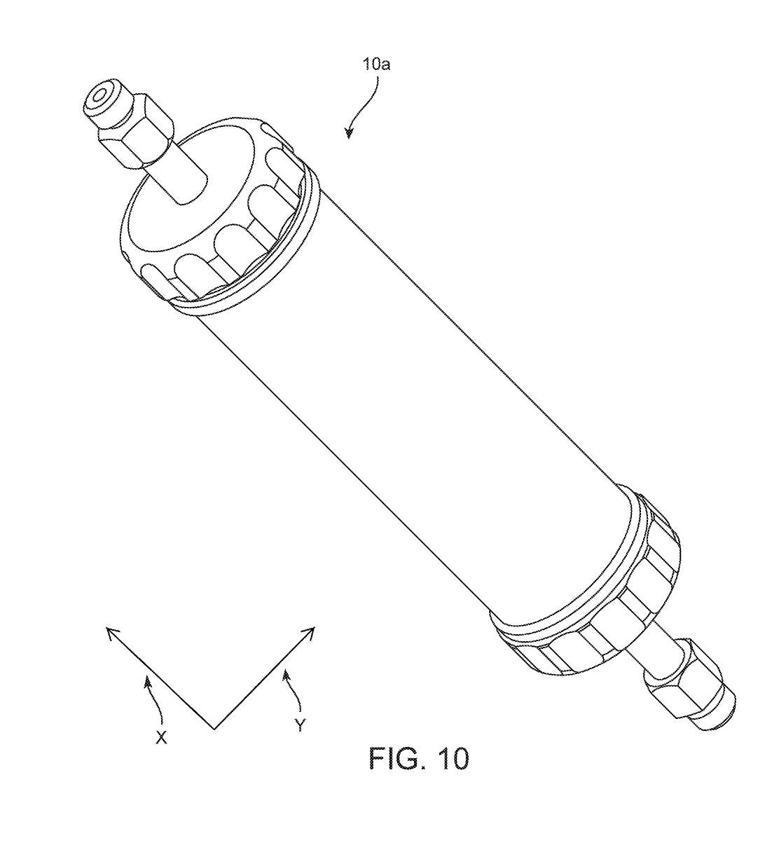

As shown in FIGS. 10-14, the device 10 may exemplify a variety of embodiments whereby each embodiment has at least two chamber tubes 30 and at least one flat filtering disk 50, 52, 54, 56, 58. Because flat filtering disks 50, 52, 54, 56, 58 are sandwiched between chamber tubes 30, the device 10 is required to have one more chamber tube 30 than flat filtering disks 50, 52, 54, 56, 58. In other words, if the device 10 has two flat filtering disks 50, 52, 54, 56, 58, then the device 10 will have three chamber tubes 30 so that the flat filtering disks 50, 52, 54, 56, 58 may be sandwiched by chamber tubes on each side. If the device 10 has three flat filtering disks 50, 52, 54, 56, 58, then the device 10 will have four chamber tubes 30. Such configurations will provide flexibility for sizing fat aspirate particles.

Specific embodiments are now described. As shown in FIG. 10, the device 10a includes a first chamber tube 30a and a second chamber tube 30b each slideably received within the hollow inner portion 14. The first and second chamber tubes 30a, 30b are hollow and each have a chamber sidewall 34a, 34b, a first portion 36a, 36b and a second portion 38a, 38b each having end walls 40a, 40b radially extending from the chamber sidewall 34a, 34b whereby the end walls 40a, 40b are arranged and configured to receive a seal 42a 42b in each of the first and second portions 36a, 36b, 38a, 38b, and a middle portion 44a, 44b located between the first and second portions 36a, 36b, 38a, 38b. The first and second chamber tubes 30a, 30b each have a plurality of tube apertures 46a, 46b located in the middle portion 44a, 44b and radially spaced apart from each other. The first chamber tube 30a being located adjacent the first cap 22, and the second chamber tube 30b being located between the first chamber tube 30a and the second cap 26. Additionally, the device 10a may include a first flat filtering disk 50 slideably received within the hollow inner portion 14 and located between the first and second chamber tubes 30a, 30b. The first flat filtering disk 50 may include a first plurality of disk apertures 60 each substantially defining a first aperture diameter 70. Because the device 10a only includes one flat filtering disk 50, the device 10a may omit side luer ports because the outlet luer port 28 may serve as the egress point. Furthermore, the device 10a may include an inlet luer port tube 25 coupled to the inlet luer port 24, an outlet luer port tube 29 coupled to the outlet luer port 28.

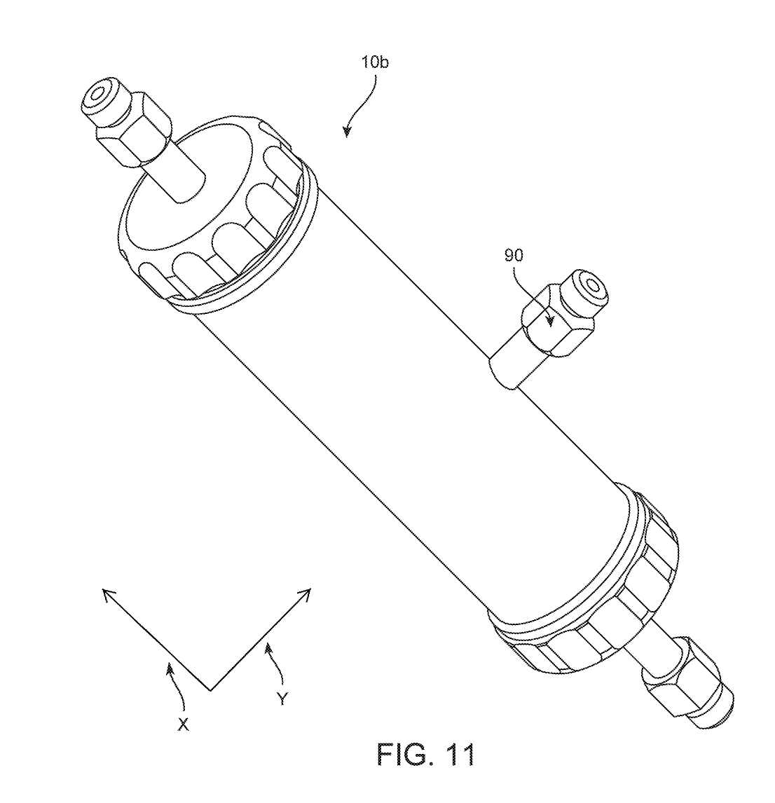

As shown in FIG. 11, another embodiment, device 10b, includes the components of device 10a and further includes a third chamber tube 30c slideably received within the hollow inner portion 14. The third chamber tube 30c is hollow and has a chamber sidewall 34c, a first portion 36c and a second portion 38c each have end walls 40c radially extending from the chamber sidewall 34c whereby the end walls 40c are arranged and configured to receive a seal 42c in each of the first and second portions 36c, 38c, and a middle portion 44c located between the first and second portions 36c, 38c. The third chamber tube 30c may also have a plurality of tube apertures 46c located in the middle portion 44c and radially spaced apart from each other. The third chamber tube 30c may be located between the second cap 26 and the second chamber tube 30b.

With respect to device 10b, the first luer port 80 may be in direct fluid communication with an aperture of the plurality of tube apertures 46b of the second chamber tube 30b. As such, the first luer port 80 may allow fat aspirate particles to travel through the first flat filtering disk 50 and then egress the device 10b via the aperture of the plurality of tube apertures 46b and the first side luer port 90, or egress at the outlet luer port 28.

The device 10b may also include a second flat filtering disk 52 slideably received within the hollow inner portion 14 and located between the second and third chamber tubes 30b, 30c. The second flat filtering disk 52 may have a second plurality of disk apertures 62 each substantially defining a second aperture diameter 72. The inlet luer port 24 and the outlet luer port 28 may both extend along the first direction X, and the first luer port 80 may thereby extend along a second direction Y that is perpendicular to the first direction X. As well, the device 10b may include a first luer port tube 90 coupled to the first luer port 80.

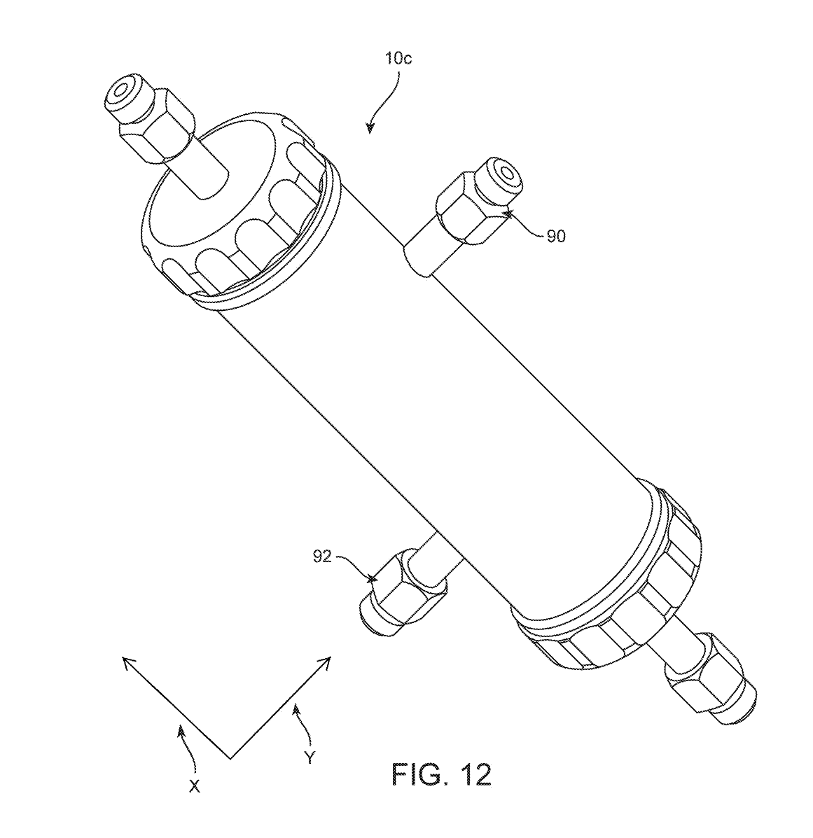

Now with respect to FIG. 12, yet another embodiment, device 10c, includes the components of device 10b and further includes a fourth chamber tube 30d slideably received within the hollow inner portion 14. Like the others, the fourth chamber tube 30d is hollow and has a chamber sidewall 34d, a first portion 36d and a second portion 38d each having end walls 40d radially extending from the chamber sidewall 34d whereby the end walls 40d are arranged and configured to receive a seal 42d in each of the first and second portions 36d, 38d, and a middle portion 44d located between the first and second portions 36d, 38d. The fourth chamber tube 30d may have a plurality of tube apertures 46d located in the middle portion 44d and radially spaced apart from each other. The fourth chamber tube 30d being located between the second cap 26 and the third chamber tube 30c.

Device 10c also includes a third flat filtering disk 54 slideably received within the hollow inner portion 14 and located between the third and fourth chamber tubes 30c, 30d. The third flat filtering disk 54 may have a third plurality of disk apertures 64 each substantially defining a third aperture diameter 74. Accordingly, the device 10c also includes a second luer port 82 located on the sidewall 16 and spaced from the first luer port 80. The second luer port 82 is in direct fluid communication with an aperture of the plurality of tube apertures 46c of the third chamber tube 30c. Naturally, the second luer port 82 may allow fat aspirate particles to travel through the first and second flat filtering disks 50, 52 and then egress the device 10c via the aperture of the plurality of tube apertures 46c and the second side luer port 92, or egress at the first luer port 90 or the outlet luer port 28. As shown in FIG. 12, both the first and second luer ports 80, 82 extend along the second direction Y. Furthermore, the first and second luer ports 80, 82 may be located on opposite sides of the tube 12 and thereby extend away from each other. Additionally, the device 10b may include a second luer port tube 92 coupled to the second luer port 82.

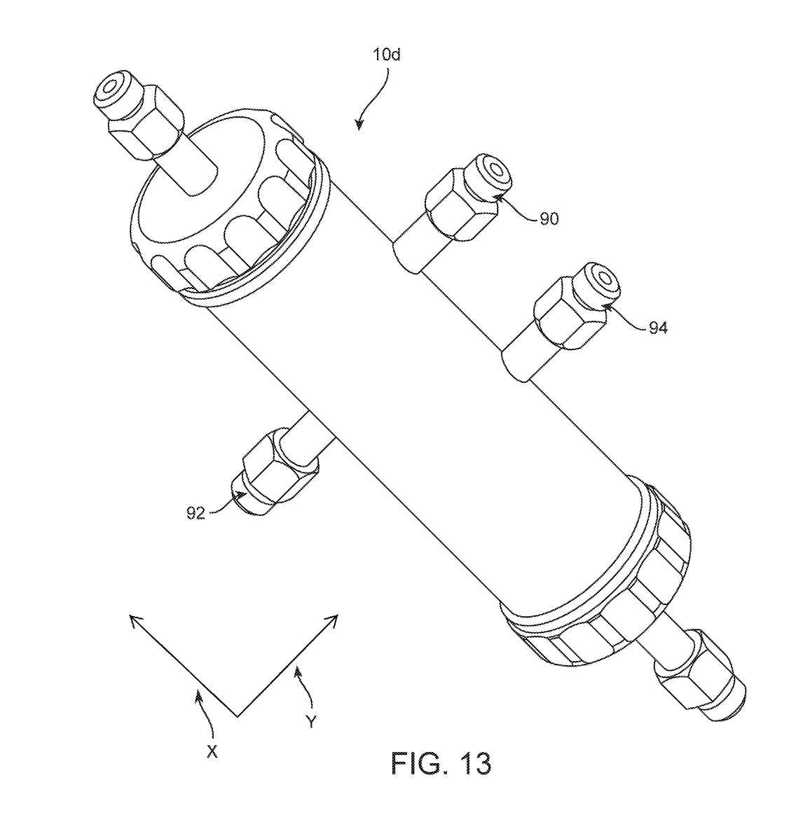

As shown in FIG. 13, device 10d includes the components of device 10c and also includes a fifth chamber tube 30e slideably received within the hollow inner portion 14. The fifth chamber tube 30e is also hollow and has a chamber sidewall 34e, a first portion 36e and a second portion 38e each having end walls 40e radially extending from the chamber sidewall 34e whereby the end walls 40e are arranged and configured to receive a seal 42e in each of the first and second portions 36e, 38e. The fifth chamber tube 30e also includes a middle portion 44e located between the first and second portions 36e, 38e. The fifth chamber tube 30e may have a plurality of tube apertures 46e located in the middle portion 44e and radially spaced apart from each other. The fifth chamber tube 30e may be located between the second cap 26 and the fourth chamber tube 30d.

Accordingly, device 10d also includes a fourth flat filtering disk 56 slideably received within the hollow inner portion 14 and located between the fourth and fifth chamber tubes 30d, 30e. The fourth flat filtering disk 56 may have a fourth plurality of disk apertures 66 each substantially defining a fourth aperture diameter 76. Device 10d also includes a third luer port 84 located on the sidewall 16 and spaced from the first and second luer ports 80, 82. The third luer port 84 being in direct fluid communication with an aperture of the plurality of tube apertures 46d of the fourth chamber tube 30d. As such, the third luer port 84 may allow fat aspirate particles to travel through the first, second, and third flat filtering disks 50, 52, 54 and then egress the device 10d via the aperture of the plurality of tube apertures 46d and the third side luer port 94, or egress at the first luer port 90, second luer port 92, or the outlet luer port 28. Moreover, as shown in FIG. 13, the first, second, and third luer ports 80, 82, 84 all extend along a second direction Y. Even still, the device 10c may include a third luer port tube 94 coupled to the third luer port 84.

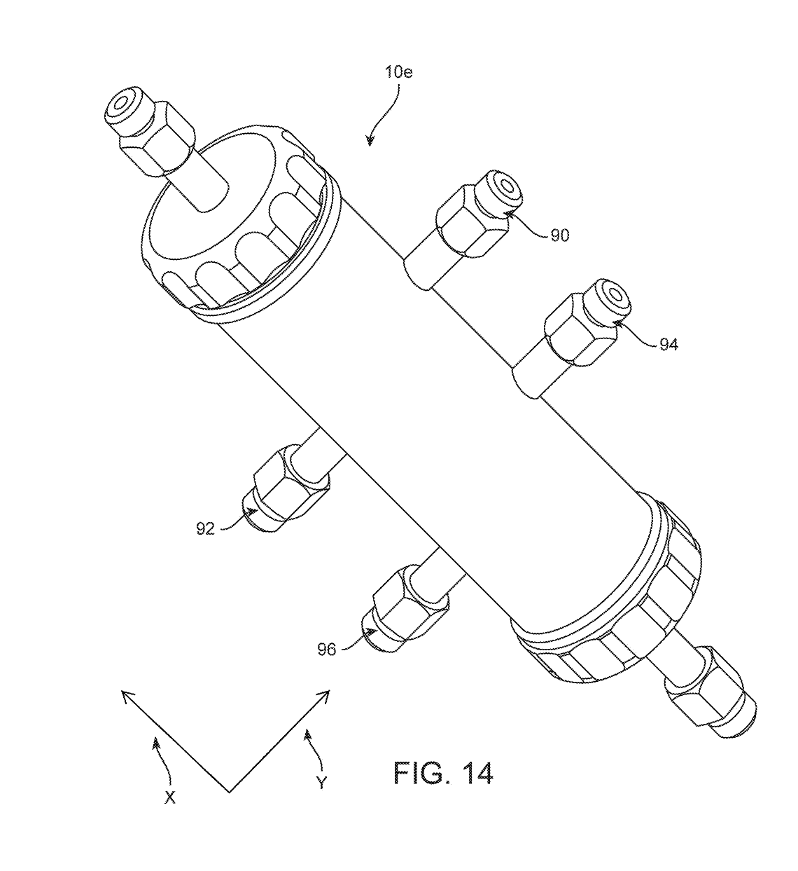

With reference to FIG. 14, device 10e includes a sixth chamber tube 30f slideably received within the hollow inner portion 14. Once again, the sixth chamber tube 30f is hollow and also has a chamber sidewall 34f, a first portion 36f and a second portion 38f each having end walls 40f radially extending from the chamber sidewall 34f whereby the end walls 40f are arranged and configured to receive a seal 42f in each of the first and second portions 36f, 38f. The chamber tube 30f also includes a middle portion 44f located between the first and second portions 36f, 38f. The sixth chamber tube 30f also has a plurality of tube apertures 46f located in the middle portion 44f and radially spaced apart from each other. The sixth chamber tube 30f is located between the second cap 26 and the fifth chamber tube 30e.

As such, the device 10e also includes a fifth flat filtering disk 58 slideably received within the hollow inner portion 14 and located between the fifth and sixth chamber tubes 30e, 30f. The fifth flat filtering disk 58 has a fifth plurality of disk apertures 68 each substantially defining a fifth aperture diameter 78. As well, the device 10e comprises a fourth luer port 86 located on the sidewall 16 and spaced from the first, second, and third luer ports 80, 82, 84. Furthermore, the device 10e may include a fourth luer port tube 96 coupled to the fourth luer port 86.

While this disclosure has described and illustrated embodiments having five flat filtering disks 50, 52, 54, 56, 58 and six chamber tubes 30a, 30b, 30c, 30d, 30e, 30f, it should be appreciated that this not limiting in anyway. The device may include greater than five flat filtering disks and greater than six chamber tubes. For example, some embodiments may include nine flat filtering disks and ten chamber tubes.

The fourth luer port 86 may be in direct fluid communication with an aperture of the plurality of tube apertures 46e of the fifth chamber tube 30e. As shown in FIG. 14, the first, second, third, and fourth luer ports 80, 82, 84, 86 all extend along the second direction Y. As expected, the fourth luer port 86 may allow fat aspirate particles to travel through the first, second, third, and fourth flat filtering disks 50, 52, 54, 56 and then egress the device 10e via the aperture of the plurality of tube apertures 46e and the fourth side luer port 96, or egress at the first luer port 90, second luer port 92, third luer port 94, or the outlet luer port 28.

Furthermore, it should be appreciated that the first, second, third, and fourth luer ports 80, 82, 84, 86 may be located along any portion of the tube 12 and extend in any direction with respect to each other. For example, in some embodiments, the second and fourth luer ports 82, 86 are located on an opposite side of the sidewall 16e of the tube 12e with respect to the first and third luer ports 80, 84.

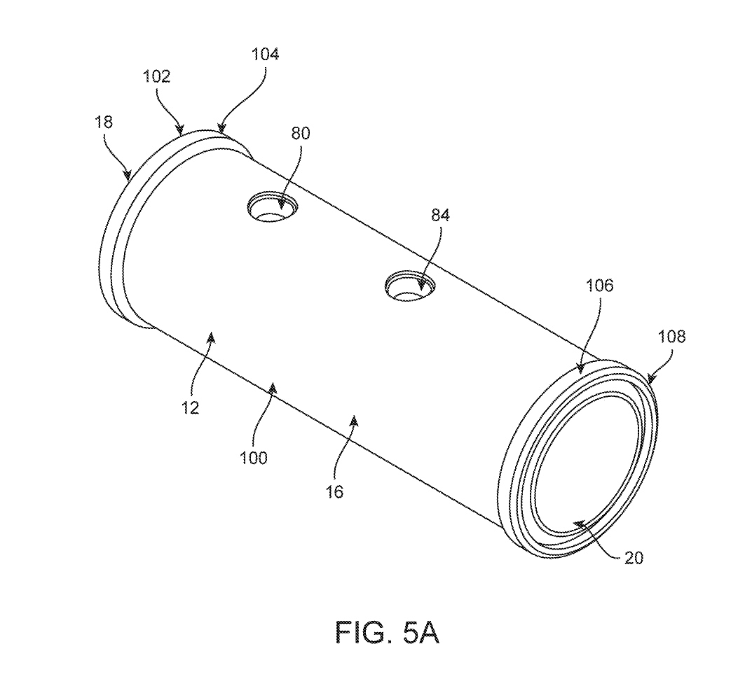

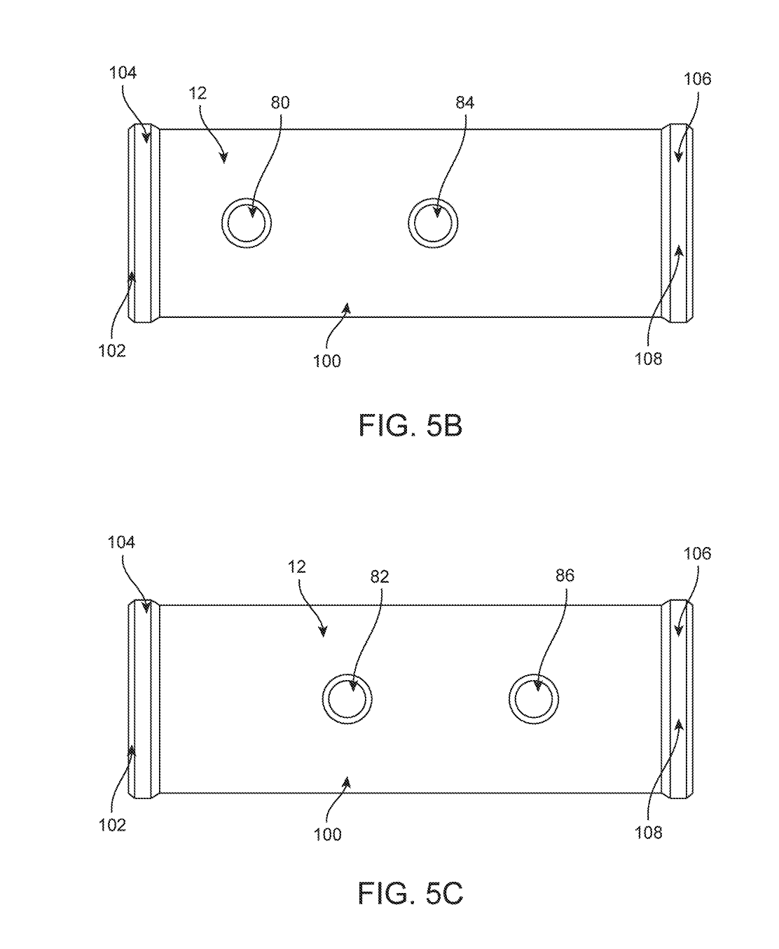

The components listed throughout this disclosure may be coupled together via a variety of connection types. As shown in FIGS. 5a, 5b, and 5c, the device 10 further includes a first lip 102 located adjacent the first open end 18. The first lip 102 may thereby be arranged and configured to receive the first cap 22. The first lip 102 may define a first lip diameter 104 that is greater than a first diameter 100 of the tube 12. Additionally, the device 10 may include a second lip 106 located adjacent the second open end 20. The second lip 106 may be arranged and configured to receive the second cap 26. The second lip 106 may also define a second lip diameter 108 that is greater than the first diameter 100 of the tube 12. In some embodiments, the first and second lips 102, 106 are each arranged and configured to receive the first and second caps 22, 26, respectively, via a snap fit or a thread fit. In many embodiments, the first lip diameter 104 is substantially equal to the second lip diameter 108.

Additionally, the device 10 may be equipped with a plurality of seals 42 coupled to the first and second portions 36, 38 of the first, second, third, fourth, fifth, and sixth chamber tubes 30a, 30b, 30c, 30d, 30e, 30f. The plurality of seals 42 are arranged and configured to seal against an outer surface of the first, second, third, fourth, fifth, and sixth chamber tubes 30a, 30b, 30c, 30d, 30e, 30f and an inner sidewall of the tube 12 to prevent liquid and fat from passing across a seal of the plurality of seals 42. This may ensure that all fat and liquid is forced to pass through the flat filtering disks 50, 52, 54, 56, 58 for appropriate sizing. In some embodiments, the plurality of seals 42 comprises a plurality of O-rings.

### Filter Tube Embodiment(s)

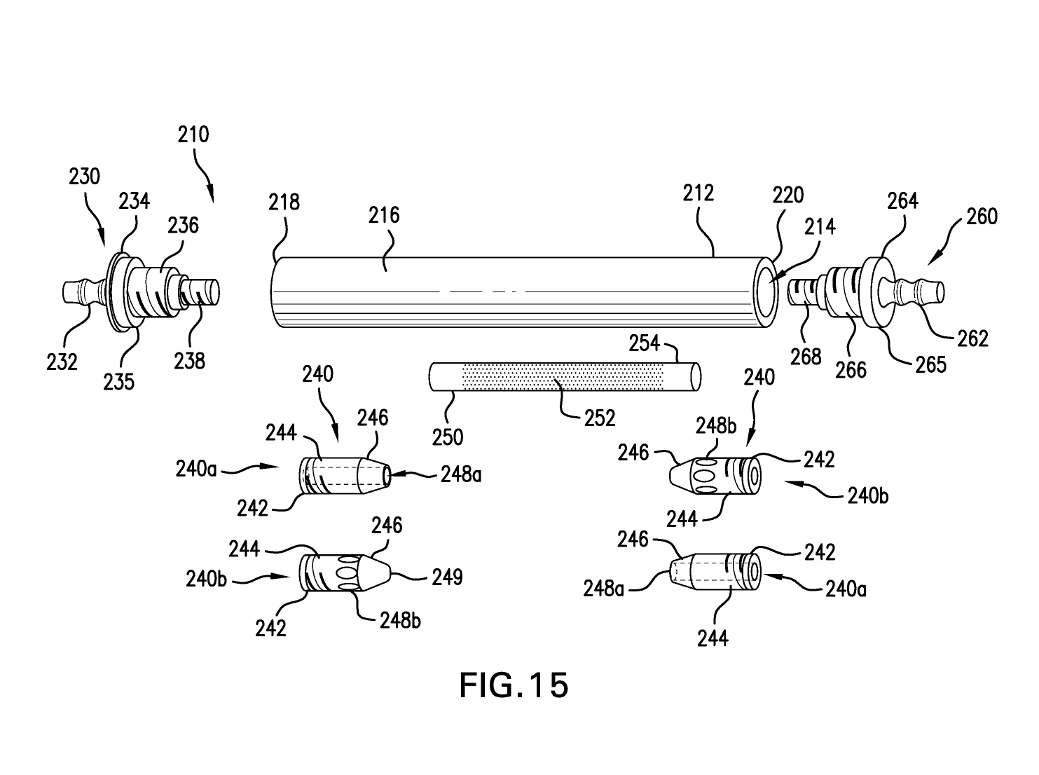

FIG. 15 is a diagram illustrating a further embodiment of an adipose tissue sizing device 210, according to some embodiments. As shown in FIG. 15, the adipose tissue sizing device 210 includes a tube 212 that has a hollow inner portion 214, a sidewall 216 radially extending around the hollow inner portion 214, a first end 218, and a second end 220 that is opposite the first end 218. An inlet interface 230 is configured to detachably connect to the first end 218. In the example shown in FIG. 15, the inlet interface 230 includes a hose barb 232 adapted to be connected to a hose or tubing (not shown in FIG. 15) through which adipose tissue material may be provided. In other examples, the inlet interface may include a tubing interface, a threaded connection, or a luer lock connection to connect to a hose or tubing, for example. The inlet interface 230 also includes a nut portion 234, an O-ring 235, a male threaded portion 236 for threaded connection to the first end 218 of the tube 212, and a male threaded portion 238 for threaded connection to a filter interface nut 240 inside the inner portion 214 of the tube 212. A through-passage, which may be of substantially uniform diameter, is formed through the inlet interface 230 from the hose barb 232 to the male threaded portion 238. Similarly, an outlet interface 260 is configured to detachably connect to the second end 220. In the example shown in FIG. 15, the outlet interface 260 includes a hose barb 262 adapted to be connected to a hose or tubing (not shown in FIG. 15) through which adipose tissue material may pass. In other examples, the outlet interface may include a tubing interface, a threaded connection, or a luer lock connection to connect to a hose or tubing, for example. The outlet interface 260 also includes a nut portion 264, an O-ring 265, a male threaded portion 266 for threaded connection to the second end 220 of the tube 212, and a male threaded portion 268 for threaded connection to a filter interface nut 240 inside the inner portion 214 of the tube 212. A through-passage, which may be of substantially uniform diameter, is formed through the outlet interface 260 from the hose barb 262 to the male threaded portion 268.

A filter assembly, in the form of filter tube 250, is positionable inside the hollow inner portion 214 of the tube 212, and is connectable to the inlet interface 230 via filter interface nut 240, and to the outlet interface 260 via another filter interface nut 240. The filter interface nut 240 includes a nut portion 242, a female threaded portion 244 configured to mate with the male threaded portion 238 of the inlet interface 230 (or the male threaded portion 268 of the outlet interface 260), and a nipple portion 246 configured to fit together with the filter tube 250, such as by a friction fit in some embodiments.

Two alternative versions of the filter interface nut 240 are shown in FIG. 15, and each will be described below with respect to the connected to the inlet interface 230. The first version of the filter interface nut 240a shows a through-passage 248a, which may be of substantially uniform diameter, that is formed through the filter interface nut 240 from the male threaded portion 238 of the inlet interface 230 to a central outlet of the nipple portion 246. The through-passage 248a is configured to allow adipose tissue particles received through the inlet interface 230 to pass into the interior of the filter tube 250. The second version of the filter interface nut 240b shows apertures 248b that are configured to allow adipose tissue particles received through the inlet interface 230 to pass through the apertures 248b upstream of the nipple portion 246 into the radial space between the filter tube 250 and the sidewall 216 of the tube 212. In this version, the distal end 249 of the nipple portion 246 is closed, so that adipose tissue particles cannot pass through it, but instead pass through the apertures 248b.

The adipose tissue sizing device 210 is configured so that complementary filter interface nut versions are employed at the inlet interface 230 and the outlet interface 260. That is, in an embodiment where the first version of the filter interface nut 240a is employed at the inlet interface 230, the second version of the filter interface nut 240b is employed at the outlet interface 260. Conversely, in an embodiment where the second version of the filter interface nut 240b is employed at the inlet interface 230, the first version of the filter interface nut 240a is employed at the outlet interface 260.

In the embodiment employing the first version of the filter interface nut 240a at the inlet interface 230, adipose tissue particles received through the inlet interface 230 pass through the through-passage 248a into the interior of the filter tube 250, and then pass from the interior of the filter tube 250 through the apertures 252 of the filter tube 250 into the radial space between the filter tube 250 and the sidewall 216 of the tube 212. In this embodiment, the second version of the filter interface nut 240b is provided at the outlet interface 260, which closes the distal end 254 of the filter tube 250 (via closed nipple portion 246), so that the adipose tissue particles are forced to pass from the interior of the filter tube 250 through the apertures 252 of the filter tube 250 into the radial space between the filter tube 250 and the sidewall 216 of the tube 212. The adipose tissue particles are effectively “filtered” by the apertures 252 of the filter tube 250 to a size that is determined by the size of the apertures 252, and then may exit from the second end 220 of the tube 212 through apertures 248b in filter interface nut 240b, and into/through outlet interface 260.

In the embodiment employing the second version of the filter interface nut 240b at the inlet interface 230, adipose tissue particles received through the inlet interface 230 pass through the apertures 248b upstream of the closed nipple portion 246 into the radial space between the filter tube 250 and the sidewall 216 of the tube 212, and then pass from this radial space region exterior to the filter tube 250 through the apertures 252 of the filter tube 250 into the interior of filter tube 250. In this embodiment, the first version of the filter interface but 240a is provided at the outlet interface 260, which results in the distal end 254 of the filter tube 250 being open by virtue of the open through-passage 248a in the nipple portion 246. The adipose tissue particles are effectively “filtered” by the apertures 252 of the filter tube 250 to a size that is determined by the size of the apertures 252, and then may exit from the open distal end 254 of the filter tube 250 through the through-passage 248a of filter interface nut 240, and out the second end 220 of the tube 212 into/through outlet interface 260.

The filter interface nut 240 may be composed of any of a number of different materials. In one example, the filter interface nut 240 may be made of polyether ether ketone (PEEK). In another example, the filter interface nut 240 may be made of stainless steel. Other materials could also be used to make the filter interface nut 240 in other embodiments.

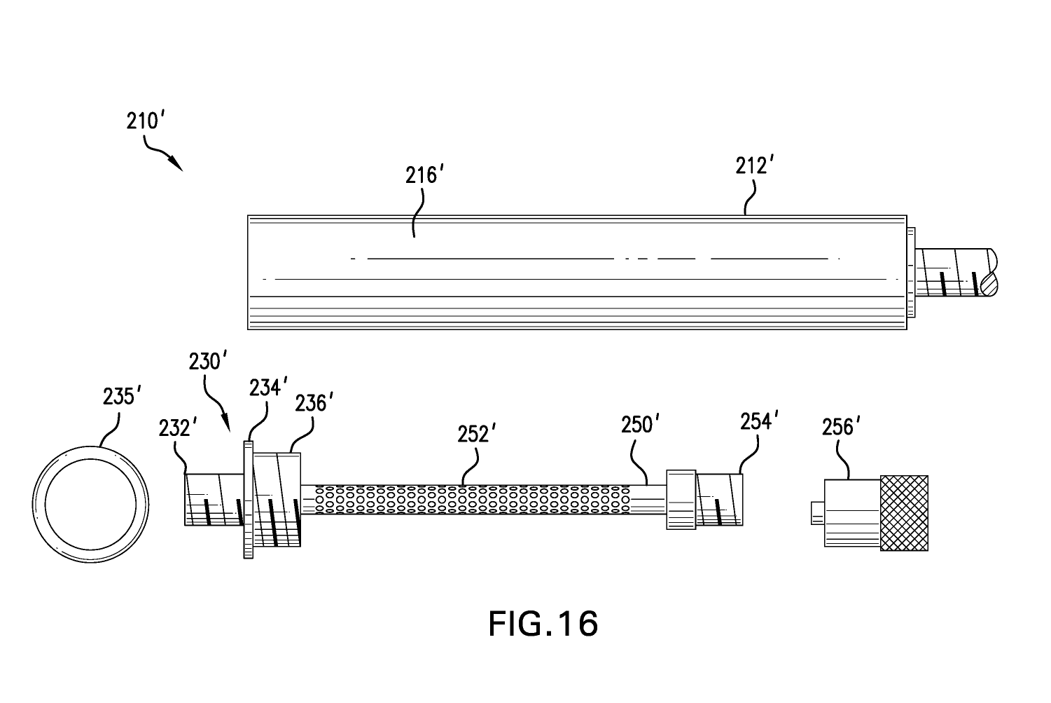

FIG. 16 is an illustration of another embodiment of an adipose tissue sizing device 210′. As shown in FIG. 16, the adipose tissue sizing device 210′ is similar to the adipose tissue sizing device 210 shown in FIG. 15, and includes similar components, except that the adipose tissue sizing device 210′ has a filter tube 250′ that is fixedly connected to the inlet interface 230′, such as by a luer lock or by welding, for example. Filter interface nut 240 is therefore not used in this embodiment. In the example shown in FIG. 16, the inlet interface 230′ is configured to allow adipose tissue particles to pass into the interior of the filter tube 250′, and the distal end 254′ of the filter tube 250′ is closed by end cap 256′, so that the adipose tissue particles are forced to pass from the interior of the filter tube 250′ through the apertures 252′ of the filter tube 250′ into the radial space between the filter tube 250′ and the sidewall 216′ of the tube 212′. The adipose tissue particles are effectively “filtered” by the apertures 252′ of the filter tube 250′ to a size that is determined by the size of the apertures 252, and then may exit from the second end 220′ of the tube 212′ via a luer port 260′.

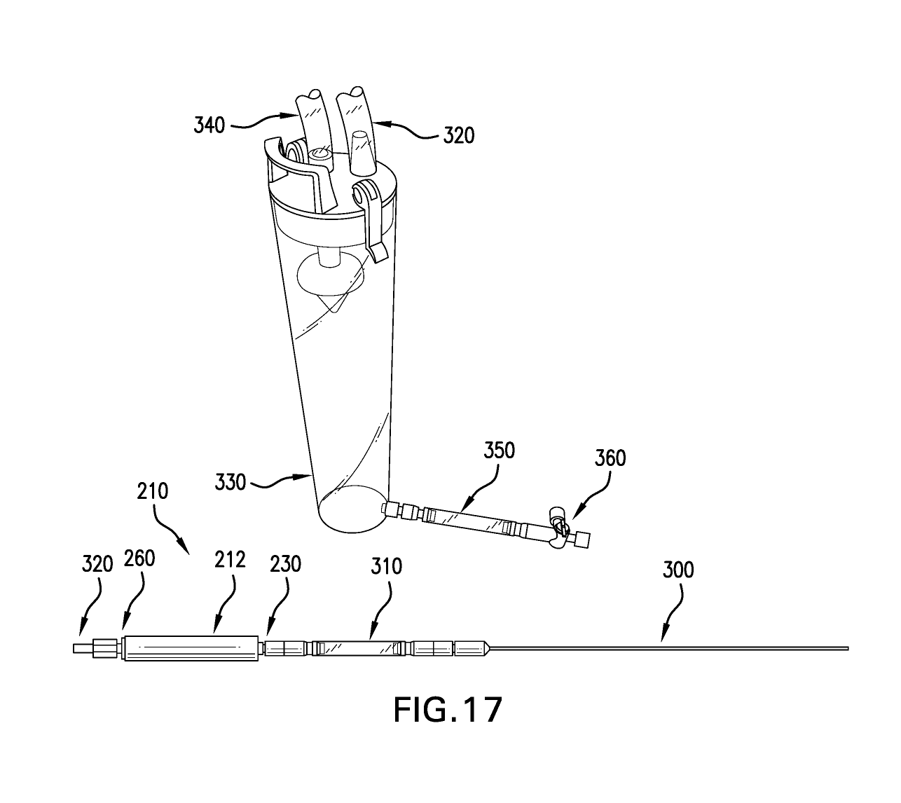

In operation, the adipose tissue sizing device 210 may be used in a number of different methods or applications. In one example, the adipose tissue sizing device 210 may be used in a two-stage liposuction process, as illustrated in FIG. 17. In such a process, a tissue harvesting tool 300, such as a cannula (illustrated in FIG. 17), may be employed to remove tissue from a selected site in a patient. The tissue harvesting tool may be connected to a first end of a length of plastic tubing 310, through which suction is applied, to cause the harvested tissue particles to travel through the tubing 310 toward a second opposite end, where the inlet interface 230 is connected (such as via a hose barb 232, as shown in FIG. 15). In another embodiment, the length of plastic tubing could be omitted, and the tissue harvesting tool/cannula 300 could be connected directly to the inlet interface 230, such as via a threaded interface or a luer lock, for example. The harvested tissue particles then pass through the inlet interface 230 (and the filter interface nut 240 in the embodiment shown in FIG. 15) to be “filtered” by the filter tube 250 (FIG. 15) inside the tube 212, to a size that is determined by the size of the apertures of the filter tube 250. The resized tissue particles then pass out of the tube 212, through the outlet interface 260 and a length of plastic tubing 320, to a container 330 (with the length of tubing 320 between outlet interface 260 and the input to the container 330 not shown, for ease of illustration in FIG. 17). The container 330 also has an input 340 connected to an aspirator/vacuum pump (not shown, for simplicity), which provides the suction for aspiration of tissue particles through the system. The container 330 also has an output connected via tubing 350 to a stopcock 360, which is operable to allow access to and/or drainage of the container 330. In one example, the stopcock 360 is closable when the container 330 is filled, to prevent fluid or contamination from reaching the aspirator pump tubing and the pump itself. The resulting stored harvested tissue particles (in container 330) have a size that is uniform and controlled by the size of the apertures 252 of the filter tube 250 (FIG. 15). This can have a number of benefits. For example, a surgeon may use a tissue harvesting tool that is configured to harvest relatively large tissue particles, which is generally easier than harvesting smaller tissue particles, and those harvested tissue particles may be resized by the filter tube 250 of the tissue sizing device 210 to a smaller size that is more desirable for future use. The uniformity of the size of the harvested tissue particles may also be significantly improved by use of the tissue sizing device 210. Other benefits may be apparent to those skilled in the art.

In other examples of operation of the adipose tissue sizing device 210, adipose tissue particles may be input through inlet interface 230 from a syringe or another storage container, instead of being directly input from a tissue harvesting procedure. Aspirating pressure to force the movement of the adipose tissue particles may be provided by a powered aspirator/vacuum pump, by operation of one or more syringes, or by other methods.

The apertures 252 may be formed in the filter tube 250 by laser drilling in some embodiments. Example sizes/diameters of the apertures 252 may be as large as 4.0 millimeters, as small as 0.2 millimeters, any size/diameter in between, or sizes/diameters larger than 4.0 millimeters or smaller than 0.2 millimeters, depending on the application in which the adipose tissue sizing device 210 is used.

In one example, the tube 212 may have an outer diameter of about 0.75 inches (about 19.05 millimeters), and the filter tube 250 may have an outer diameter of about 0.259 inches (about 6.58 millimeters). In other examples, the tube 212 and the filter tube 250 may have larger or smaller radial dimensions. In some embodiments, the tube 212 and the filter tube 250 are composed of stainless steel.

In some embodiments, the tube 212 is designed to be a reusable components, while the filter tube 250 is designed to be a single-use, disposable component. Filter interface nut 240 may also be a reusable component in some embodiments. In certain embodiments, filter tube 250 may also be a reusable component. In this context, components described as reusable are capable of being cleaned and sterilized multiple times, such as be a sterilizing autoclave, by enzyme treatment, or by other methods.

### Interpretation

The term “approximately” means that something is almost, but not completely, accurate or exact; roughly. Additionally, the term “substantially” means to a great or significant extent; for the most part, essentially.

None of the steps described herein is essential or indispensable. Any of the steps can be adjusted or modified. Other or additional steps can be used. Any portion of any of the steps, processes, structures, and/or devices disclosed or illustrated in one embodiment, flowchart, or example in this specification can be combined or used with or instead of any other portion of any of the steps, processes, structures, and/or devices disclosed or illustrated in a different embodiment, flowchart, or example. The embodiments and examples provided herein are not intended to be discrete and separate from each other.

The section headings and subheadings provided herein are nonlimiting. The section headings and subheadings do not represent or limit the full scope of the embodiments described in the sections to which the headings and subheadings pertain. For example, a section titled “Topic 1” may include embodiments that do not pertain to Topic 1 and embodiments described in other sections may apply to and be combined with embodiments described within the “Topic 1” section.

Some of the devices, systems, embodiments, and processes use computers. Each of the routines, processes, methods, and algorithms described in the preceding sections may be embodied in, and fully or partially automated by, code modules executed by one or more computers, computer processors, or machines configured to execute computer instructions. The code modules may be stored on any type of non-transitory computer-readable storage medium or tangible computer storage device, such as hard drives, solid state memory, flash memory, optical disc, and/or the like. The processes and algorithms may be implemented partially or wholly in application-specific circuitry. The results of the disclosed processes and process steps may be stored, persistently or otherwise, in any type of non-transitory computer storage such as, e.g., volatile or non-volatile storage.

The various features and processes described above may be used independently of one another, or may be combined in various ways. All possible combinations and subcombinations are intended to fall within the scope of this disclosure. In addition, certain method, event, state, or process blocks may be omitted in some implementations. The methods, steps, and processes described herein are also not limited to any particular sequence, and the blocks, steps, or states relating thereto can be performed in other sequences that are appropriate. For example, described tasks or events may be performed in an order other than the order specifically disclosed. Multiple steps may be combined in a single block or state. The example tasks or events may be performed in serial, in parallel, or in some other manner. Tasks or events may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require at least one of X, at least one of Y, and at least one of Z to each be present.

The term “and/or” means that “and” applies to some embodiments and “or” applies to some embodiments. Thus, A, B, and/or C can be replaced with A, B, and C written in one sentence and A, B, or C written in another sentence. A, B, and/or C means that some embodiments can include A and B, some embodiments can include A and C, some embodiments can include B and C, some embodiments can only include A, some embodiments can include only B, some embodiments can include only C, and some embodiments can include A, B, and C. The term “and/or” is used to avoid unnecessary redundancy.

While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein.

The following is claimed:

1. An adipose tissue sizing device, comprising:
a first tube elongate along a first direction, the first tube having a hollow inner portion, a sidewall radially extending around the hollow inner portion, a first end, and a second end opposite the first end;
an inlet interface in fluid communication with the hollow inner portion of the first tube, configured to receive adipose tissue particles, and allow the adipose tissue particles to pass into the hollow inner portion of the first tube;
a filtering assembly having a plurality of apertures therein, the filtering assembly being positioned in the hollow inner portion of the first tube in a flow pathway of the adipose tissue particles, and being configured to resize the adipose tissue particles passing through the apertures thereof according to a size of the apertures;
an outlet interface in fluid communication with the hollow inner portion of the first tube, configured to allow the resized adipose tissue particles to pass out of the hollow inner portion of the first tube.

2. The device of claim 1, wherein the filtering assembly comprises a second tube having the plurality of apertures in a radially outer surface thereof, the second tube having a first end coupled to the inlet interface and a second end coupled to the outlet interface, and being positioned in the hollow inner portion of the first tube.

3. The device of claim 2, wherein:
the inlet interface is configured to receive adipose tissue particles and direct the adipose tissue particles into an interior of the second tube;
the second end of the second tube is closed, so that the adipose tissue particles directed to the interior of the second tube pass through the apertures in the radially outer surface of the second tube into a space between the second tube and the first tube; and
the outlet interface is configured to allow the resized adipose tissue particles to pass out of the space between the second tube and the first tube in the hollow inner portion of the first tube.

4. The device of claim 2, wherein:
the inlet interface is configured to receive adipose tissue particles and direct the adipose tissue particles into a space between the second tube and the first tube;
the first end of the second tube is closed, the second end of the second tube is open, and the second end of the first tube is closed in the space between the second tube and the first tube, so that the adipose tissue particles directed to the space between the second tube and the first tube pass through the apertures in the radially outer surface of the second tube into an interior of the second tube; and
the outlet interface is configured to allow the resized adipose tissue particles to pass out of the interior of the second tube.

5. The device of claim 3, wherein the inlet interface comprises:
a first threaded portion releasably connected to the first end of the first tube;
a second threaded portion;
a filter interface nut having a third threaded portion releasably connected to the second threaded portion of the inlet interface, a nipple portion for coupling to the first end of the second tube, and a through-passage extending through the nipple portion into the interior of the second tube, in fluid communication with an input to the inlet interface.

6. The device of claim 5, wherein the outlet interface comprises:
a fourth threaded portion releasably connected to the second end of the first tube;
a fifth threaded portion; and
a filter interface nut having a sixth threaded portion releasably connected to the fifth threaded portion of the outlet interface, a nipple portion having a closed end for coupling to the second end of the second tube, and apertures located downstream of the second end of the second tube, in fluid communication with the space between the second tube and the first tube.

7. The device of claim 4, wherein the inlet interface comprises
a first threaded portion releasably connected to the first end of the first tube;
a second threaded portion;
a filter interface nut having a third threaded portion releasably connected to the second threaded portion of the inlet interface, a nipple portion having a closed end for coupling to the first end of the second tube, and apertures located upstream of the first end of the second tube, in fluid communication with the space between the second tube and the first tube.

8. The device of claim 7, wherein the outlet interface comprises:
a fourth threaded portion releasably connected to the second end of the first tube;
a fifth threaded portion;
a filter interface nut having a sixth threaded portion releasably connected to the fifth threaded portion of the outlet interface, a nipple portion for coupling to the second end of the second tube, and a through-passage extending through the nipple portion into the interior of the second tube, in fluid communication with an output of the outlet interface.

9. The device of claim 1, wherein the filtering assembly comprises a filtering disk positioned in the hollow inner portion of the first tube in the flow pathway of the adipose tissue particles, the filtering disk having a flat surface with the plurality of apertures extending therethrough that faces the flow pathway of the adipose tissue particles.

10. The device of claim 1, wherein the plurality of apertures have a diameter of 0.1-4.0 mm.

11. The device of claim 1, wherein the first tube has an outer diameter of about 0.75 inches (about 19.05 millimeters).

12. The device of claim 2, wherein the first tube has an outer diameter of about 0.75 inches (about 19.05 millimeters), and the second tube has an outer diameter of about 0.259 inches (about 6.58 millimeters).

13. The device of claim 1, wherein the inlet interface comprises a hose barb for connection to a tissue harvesting tool via tubing.

14. The device of claim 1, wherein the inlet interface comprises a threaded interface or a luer lock interface for connection to a tissue harvesting tool.

15. A dual-stage liposuction system comprising:
a tissue harvesting tool configured for harvesting adipose tissue particles from a patient;
a adipose tissue sizing device of claim 1, having the inlet interface coupled to the tissue harvesting tool for receiving the harvested adipose tissue particles;
a container coupled to the outlet interface of the adipose tissue sizing device to receive resized adipose tissue particles therefrom, the container being couplable to a pump to provide suction to aspirate the adipose tissue particles through the system.
